(19) United States
(12) Patent Application Publication
(10) Pub. No.: US 2025/0375123 A1
(43) Pub. Date: Dec. 11, 2025

(54) ANALYSIS OF GAS SAMPLES FOR DETERMINATION OF PHYSIOLOGICAL STATES AND DISEASE STATES

(71) Applicant: New York University, New York, NY (US)

(72) Inventors: Kalle Levon, New York, NY (US); Hao-Chun Chiang, New York, NY (US); Nikita Grigoryev, New York, NY (US); Freya Schnabel, New York, NY (US); Ivan Selesnick, New York, NY (US)

(21) Appl. No.: 19/180,552

(22) Filed: Apr. 16, 2025

Related U.S. Application Data
(63) Continuation of application No. 17/175,557, filed on Feb. 12, 2021, now abandoned.
(60) Provisional application No. 62/975,648, filed on Feb. 12, 2020.

(51) Int. Cl. A61B 5/08; A61B 5/097; G01N 33/00

(52) U.S. Cl. CPC A61B 5/082; A61B 5/097; G01N 33/0075

(57) ABSTRACT

Provided methods of detecting physiological states and/or disease states of an individual. Also provided are systems for detecting physiological states and/or disease states of an individual. The method are based on the response of organic semiconducting materials to one or more components of a biogas sample from an individual.

### CROSS REFERENCE TO RELATED APPLICATIONS

This application is a continuation of U.S. patent application Ser. No. 17/175,557, filed on Feb. 12, 2021, which claims priority to U.S. Provisional Patent Application No. 62/975,648, filed Feb. 12, 2020, the disclosures of which are hereby incorporated herein by reference.

### BACKGROUND OF THE DISCLOSURE

The human body releases many volatile organic compounds. These compounds typically reflect the body's current metabolic condition, cells, tissues, and microbiome. The body's metabolism consists of chemical processes that are required to sustain life. Small molecules, known as metabolites, circulate in the body and are reliable biomarkers because their profiles are changed when the body's biological system is affected due to disease, mutations, or even environmental factors.

Cancer is the second leading cause of death worldwide. It is very hard to diagnose patients early because most of the symptoms do not appear and diagnosing procedures are costly and can sometimes pose risks to the patient. Breast cancer typically is diagnosed through mammograms, MRIs, and biopsies. Mammograms can expose excessive risk of radiation, risk tumors rupturing and over diagnose breast cancer. Lung cancer can be diagnosed through X ray images and CT scans. Colonoscopies are used to detect colon cancer in patients. All these methods are time consuming and may impose some risks for patients. These methods are also typically used when there are symptoms of cancer showing on the body.

It is important to be able to detect cancer in the early stages so that proper treatment can be provided and significantly increase the chance of survival for the patient. However, most cancer-linked symptoms are not as easily recognizable unless a thorough procedure is done, such as a biopsy. These procedures happen to be very costly and can have negative health risks associated with it, hence they are mainly done when symptoms are more obviously present in the patient. Scientists have come up with a simpler and more cost effective procedure in which breath samples are taken of patients. Breath samples give an overall body blood sampling and the metabolites in breath are due to the current conditions of the body, hence it would reflect if there are new diseases. However, there are currently no efficient ways of correlating breath sample analysis to diseased physiological states.

### SUMMARY OF THE DISCLOSURE

The present disclosure provides methods of detecting physiological states and/or disease states of an individual. Also provided are systems for detecting physiological states and/or disease states of an individual.

In an aspect the method comprises obtaining and combining responses from a plurality of sensors which can detect and are specific for one or more distinct target molecules. By identification of the combination of responses generated by a biological sample, a determination of the sample disease or physiological state can be made.

In various examples, the disclosure describes how different biogas samples (such as, for example, breath samples) exhibit a different sensor response (e.g. smell), and how, for example, biogas samples are analyzed to detect, for example, physiological states and/or disease states (such as, for example, cancers and other diseases). For example, breath biopsies can also measure drug activity, drug compliance, response to therapies, etc. Individuals can be easily screened which can allow early detection of physiological states and/or disease states (e.g., cancer) and this can save lives.

In an aspect, the present disclosure provides methods of detecting physiological states and/or disease states. The methods are based on the response of organic semiconducting materials to one or more components of a biogas sample from an individual. Non-limiting examples of methods of the present disclosure are provided.

Various biogas samples may be used. A biogas sample may also referred to herein as biogas specimen. A biogas sample is obtained directly (e.g., in the case of a breath sample from an individual) or indirectly (e.g., the headspace gas from a liquid or tissue sample from an individual). Non-limiting examples of biogas samples include breath from an individual and/or a gas sample derived from (e.g., evolved from) from one or more bodily fluid(s) (such as, for example, sweat, urine, saliva, blood, and the like), cells, stool, tissue, and the like, and combinations thereof. The biogas sample may contain vapor materials such as water vapors and aqueous aerosols.

In an aspect, the present disclosure provides sensors. A sensor comprises one or more layer(s) of organic semiconducting material. The sensors may be used in a method of the present disclosure or a system of the present disclosure. Non-limiting examples of sensors of the present disclosure are provided.

In an aspect, the present disclosure provides systems. The systems can be used for detecting physiological states and/or disease states. In various examples, a system is used to carry out a method of the present disclosure. Non-limiting examples of systems of the present disclosure are provided.

The system (e.g., system comprising a sensor array) may be provided in the form of a device. The identification of the physiological and/or diseased state may be achieved without the necessity to identify the individual components of the biogas sample. The altered electrical signals from the sensors (which encodes information about the presence of the components of the biogas sample and their respective concentrations) are communicated to other components to be processed further. The readout and analysis component may be software that interprets the signals and correlates them to a particular physiological and/or disease state.

### BRIEF DESCRIPTION OF THE FIGURES

For a fuller understanding of the nature and objects of the disclosure, reference should be made to the following detailed description taken in conjunction with the accompanying figures.

FIG. 1 shows baseline of conductivity for a sensor by exposing to air and vapor of isopropyl glycol solution.

FIG. 2 shows an example of a sensor having interdigitated platinum finger electrodes with spun cast polyaniline on top.

FIG. 3 shows analysis of various chemicals. The chemical sensors measure different chemical compounds that represent different scents.

FIG. 4 shows analysis of 4 different wines. 1 is Syrah, 2 is Valpolicella Ripasso Docsuperiore, 3 is Rioja, and 4 is Noblesse Du Terroir.

FIG. 5 shows analysis (PCA map) of various samples of healthy cells, cancer cells, and mixtures thereof.

FIG. 6 shows analysis of various compounds. The chemical sensors measure different chemical compounds that represent different scents.

FIG. 7 shows U87 cell scent results.

FIG. 8 shows the effect of three gases (jasmine, benzyl acetate, and indole) on sensors of the present disclosure. The top plot shows the data obtained from utilizing a single sensor and the bottom plot shows the data obtained from utilizing three sensors. The

FIG. 9 shows data for the effect of five gases on six sensors. All the molecules are identified in various cancer breath analyses.

FIG. 10 shows an image of a sensor near a cancer cell culture.

FIG. 11 shows measurement data. The data is PANI/DNNSA i-t response to cancer cell exposure. The current was reduced when compared with a control cell solution. The measurement was performed at 37° C.

FIG. 12 shows a list of dopants. These dopants were used with the sensor provided in FIG. 2.

FIG. 13 shows a doping protocol. The PANI doping process was performed in an NMP solution. These dopants were used with the sensor provided in FIG. 2.

FIG. 14 shows an image of a sensor system of the present disclosure. The responses were from the displayed sensor system were measured with a potentiostat.

FIG. 15 shows a graphical representation of a method of the present disclosure. Multiple sensors specific for different scents may be applied. Examples shown are not quantitatively arranged.

FIG. 16 shows a graphical representation of a method of the present disclosure. The response obtained from the cells changed according to the change in the scent. As the concentration of cancer cells was increased while the concentration of healthy cells decreased, there was an increase in the scent associated with methylsulfide. When only cancerous cells were present, the change was monitored with a sensory array of the present disclosure (e.g., MultiCharged Sensor Array).

FIG. 17 shows a graphical representation of a 1D sensor (1 sensor). In this representation, the arrow describes the “directionality” of sensor and the amount of information received by a single sensor. A single sensor does not provide a “full view” of all the data.

FIG. 18 shows a graphical representation of a 2D sensor (2 sensors). In this representation, the arrows describe the “directionality” of sensors and the amount of information received by two sensor. Two sensors provide more data than a single sensor.

FIG. 19 shows a graphical representation of a 3D sensor (3 sensors). In this representation, the arrows describe the “directionality” of sensors and the amount of information received by three sensor. Three sensors provide more data than two sensors or a single sensor.

FIG. 20 shows a graphical representation of a 4D sensor (4 sensors). In this representation, the arrows describe the “directionality” of sensors and the amount of information received by four sensor. Four sensors provide more data than three sensors, two sensors, or a single sensor.

FIG. 21 shows a graphical representation of 8 sensors. In this representation, the arrows describe the “directionality” of sensors and the amount of information received by eight sensor. Eight sensors provide more data than four sensors, three sensors, two sensors, or a single sensor.

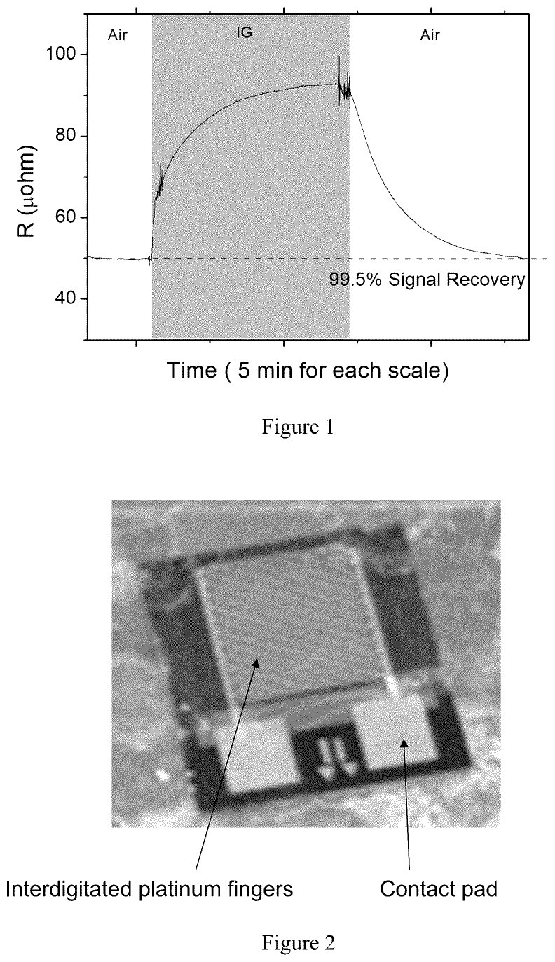

### DETAILED DESCRIPTION OF THE DISCLOSURE

Although claimed subject matter will be described in terms of certain examples and/or embodiments, other embodiments, including embodiments that do not provide all of the benefits and features set forth herein, are also within the scope of this disclosure. Various structural, logical, process step, and electronic changes may be made without departing from the scope of the disclosure.

Ranges of values are disclosed herein. The ranges set out a lower limit value and an upper limit value. Unless otherwise stated, the ranges include all values to the magnitude of the smallest value (either lower limit value or upper limit value) and ranges between the values of the stated range.

The present disclosure provides methods of detecting physiological states and/or disease states of an individual. Also provided are systems for detecting physiological states and/or disease states of an individual.

In various examples, the disclosure describes how different biogas samples (such as, for example, breath samples or volatile samples generated from any cell or tissue samples, e.g., cell culture container head space samples) exhibit a different sensor response (e.g. smell), and how, for example, biogas samples are analyzed to detect, for example, physiological states and/or disease states (such as, for example, cancers and other diseases). For example, breath biopsies can also measure drug activity, drug compliance, response to therapies, etc. Individuals can be easily screened which can allow early detection of physiological states and/or disease states (e.g., cancer) and this can aid in designing therapies.

In an aspect, the present disclosure provides methods of detecting physiological states and/or disease states. The methods are based on the response of organic semiconducting materials to one or more components of a biogas sample from an individual. Non-limiting examples of methods of the present disclosure are provided herein.

In various examples, a method for diagnosis of a physiological state and/or diseased state by analysis of a test biogas sample comprises: providing an array of sensors comprising a plurality of distinct sensors, where each distinct sensor comprises a layer of organic semiconducting material, which may be i) at least partially oxidized or ii) at least partially doped or iii) is protonated by one or more component of the test biogas sample (e.g., each individual distinct sensor may be different in terms of one or more or all of dopant, dopant concentration, or organic semiconducting material from other distinct sensors); and contacting (e.g., exposing) the array of sensors to the test biogas sample, where upon contacting (e.g., exposing) a response or responses from the array of sensors is/are generated. The response(s) correlates to the presence or absence of a physiological state and/or diseased state. In an example, if one or more specific volatile compounds are present, the distinct sensors reactive to the specific volatile compounds or a different concentration of the same compound exhibit separately detectable responses and based on the combination of detected responses, the presence or absence of the physiological state or disease state is identified.

In various examples, the instant methods use organic semiconducting materials to determine the end markers in a diversity map for sensing scents by electrochemical methods. Without intending to be bound by any particular theory, it is considered that the sensitivity of the organic conducting material is dependent on, for example, the nature of the organic semiconducting materials that may comprise a macromolecular polymer chain and a dopant. The dopant may induce a charge on the polymer chain. If the charge on the polymer is positive, the chain is a hole conductor and if the charge is negative it is an electron conductor. The dopant stays in anionic (−) or cationic (+) state opposite of the charge in the polymer chain. The chemical nature of the dopant, the degree of doping, the chemical nature of the chain with its structure (copolymer/block copolymer), its modification (substituents), and manner of mixing with other components give the tuning opportunities for controlling the sensing dimensions. Various organic semiconducting materials can be used. In various examples, the organic semiconducting material has a conductivity of 1×10⁻⁵ to 1000 S/cm², including all 1×10⁻⁶ S/cm² values and ranges therebetween.

In non-limiting examples, the organic semiconducting material is independently for each sensor chosen from organic semiconducting oligomers (which may be referred to herein as organic oligomer semiconducting materials or organic oligomeric semiconducting materials) and organic semiconducting polymers (which may be referred to herein as organic polymer semiconducting materials or organic polymeric semiconducting materials), and the like, and combination thereof. Non-limiting examples of organic semiconducting polymers include polyaniline (PANI) and substituted analogs thereof, polythiophene and substituted analogs thereof, polypyrrole and substituted analogs thereof, and the like, block copolymers comprising one or more block thereof, graft copolymers comprising one or more block thereof, network polymers thereof, and the like, and combinations thereof. Individual organic semiconducting materials may be amorphous or at least partially crystalline or at least partially amorphous

Polyaniline (PANI) is a desirable conducting polymer due, at least in part to, its facile synthesis in acidic aqueous solutions, environmental stability, inexpensive monomer, good processability, and solubility in common organic solvents, which allows it to be blended with other polymers. PANI exhibits three different oxidation states: leucoemeraldine (LEB, fully reduced), emeraldine (EB, half-oxidized), and pernigraniline (PNB, fully oxidised), however, emeraldine salt, the protonated form of EB, is the only conducting form and is usually obtained by protonation of the basic amine and imine sites in EB with strong acids. This process is reversible thus imparting pH sensitivity to PANI. Furthermore, the pH and ionic sensitivity can be tuned by doping PANI with mobile or immobile counter ions.

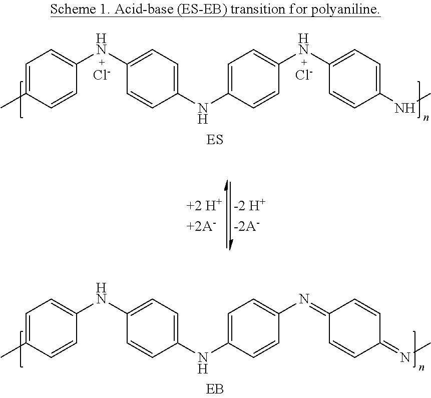

Scheme 1 shows the acid-base transition of polyaniline, which renders polyaniline pH sensitive. This is an important characteristic of PANI, especially for ammonia detection, as it deprotonates the amine groups in the emeraldine salt converting it to the emeraldine base form with a corresponding drop in conductivity of several orders of magnitude. The reaction that allows this change in conductivity is given by the reaction:

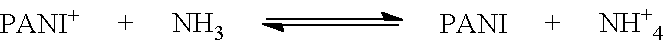

By proper selection of the dopant protonic acid, this pH sensitivity can be further increased. Polyaniline doped with camphor sulfonic acid has shown to have a pH sensitivity of around 70 mV, which is higher than 59 mV typically observed with other small counter ions like (Cl⁻ and SO₄²⁻) and that is the basis for selecting CSA over the other protonic acids for the PANI ammonia sensors.

Schemes 2 and 3 provide examples of conducting polymers of the present disclosure and methods of making such conducting polymers:

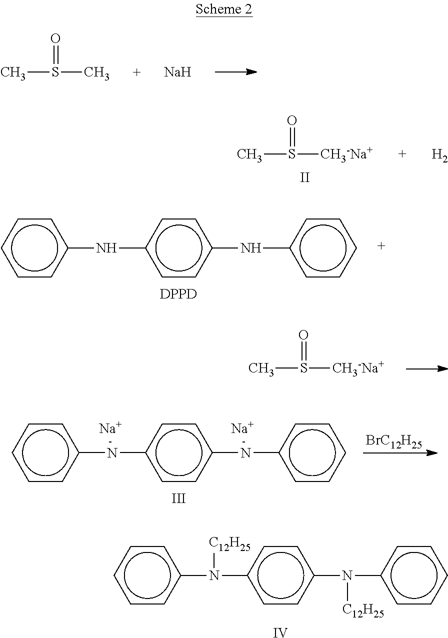

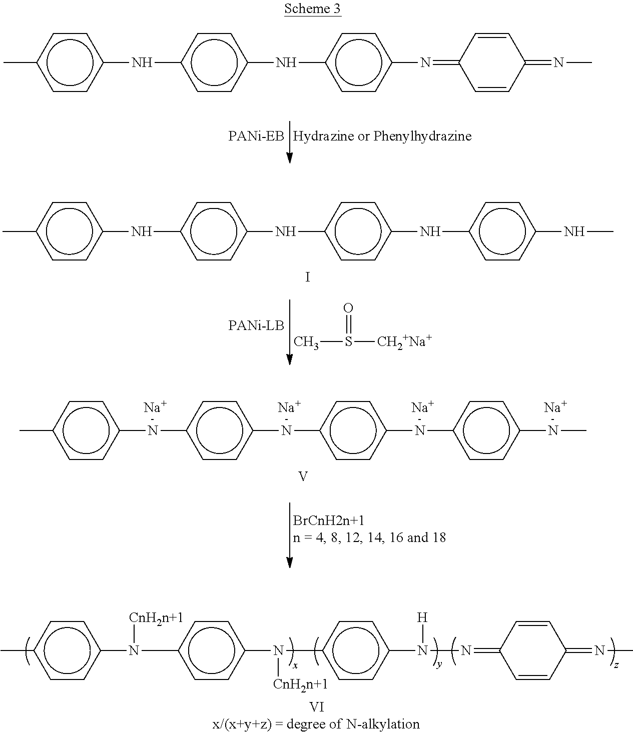

The organic semiconducting material may be formed by oxidative polymerization of monomers, such as, for example, aniline, thiophene, pyrrole, substituted analogs thereof (e.g., carboxylated, sulfonated, phosphorylated, alkylated, alkoxylated, etherified analogs thereof, or the like, or a combination thereof), and the like, and combinations thereof. The copolymers and network copolymers may comprise polymerized monomers, such as, for example, non-electroactive monomers with electroactive monomers. Non-limiting examples of non-electroactive monomers include all vinyl linked monomers, difunctional esters, alcohols, carboxylic acids, and the like.

The organic semiconducting material(s) may be a polymer or polymers that is/are modified post polymerization (e.g., sulfonated, anion exchanged, cation exchanged, or the like, or a combination thereof).

The organic semiconducting material(s) may be an oligomer or oligomers or polymer or polymers having various molecular weights. In various examples, the organic semiconducting material is an oligomer or oligomers having a molecular weight of 10 to 500 g/mol, including all 0.1 g/mol values and ranges therebetween, and/or a polymer having a molecular weight of 200 to 500,000 g/mol, including all 0.1 g/mol values and ranges therebetween. The molecular weight (M_{w} and/or Mₙ) may be determined by methods known in the art. Non-limiting examples of such methods include size exclusion chromatography, which may be carried out by comparison to one or more polystyrene standard(s)).

The organic semiconductor materials may have various secondary structure. In various examples, the individual organic semiconducting material(s) is/are amorphous or at least partially crystalline or amorphous.

The organic semiconductor materials may be doped with various dopants.

Non-limiting examples of dopants include oxidants, acids (e.g., mineral acids (such as, for example, hydrochloric acid, sulfonic acid, nitric acid, and the like, organic acids (such as, for example, sulfonic acids (e.g., camphor sulfonic acid, p-toluene sulfonic acid), alkylated (e.g., C₁-C₂₀ alkyl) acids, aryl acids, phosphonic acids, and the like), polymers (such as, for example, sulfonated polystyrene, polyphosphazines, self-doped sulfonated polyanilines, and the like), and the like, and combinations thereof. It may be desirable to use one or more oxidant(s) as dopants for polythiophenes and polypyrroles.

The organic semiconductor material may be a self-doped organic conducting material. Non-limiting examples of self-doped organic conducing materials include poly(2-methoxyaniline-5-phosphonic acid) (PMAP), poly(2-methoxyaniline-5-sulfonic acid, and the like), and copolymers (e.g., block copolymers, graft copolymers, and the like), network polymers thereof.

The dopant(s) may be present in individual organic semiconductor materials in various amounts. For example, the dopant concentration is independently for each sensor 2 to 50 weight % (based on the total weight of the organic semiconducting material and dopant(s)), including all 0.1 weight % values and ranges therebetween.

The doping is an oxidative process creating charged units with mobile delocalized holes or electrons or a protonation in which already oxidized form of polymers is protonated creating the hole transport process along the chains.

Non-limiting examples of dopants and doping strategies include the following: Hydrochloric acid (an example of HCl doping follows:

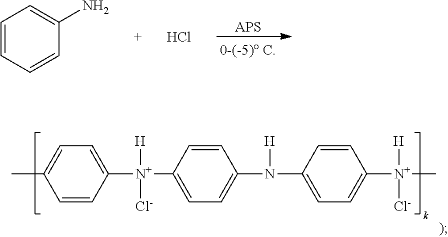

Camphor sulphonic acid (which can be + or − chiral or racemic). A non-limiting example of a camphor sulphonic acid is 10-camphorsulphonic acid;
p-toluene sulphonic acid; dodecyl benzene sulphonic acid (an example of dodecyl benzene sulphonic acid doping follows:

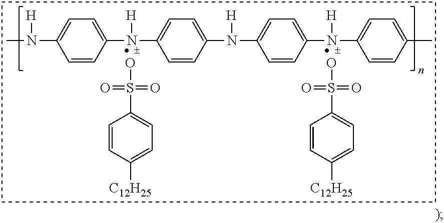

dinonyl naphthalene sulphonic acid (an example of dodecyl benzene sulphonic acid doping follows:

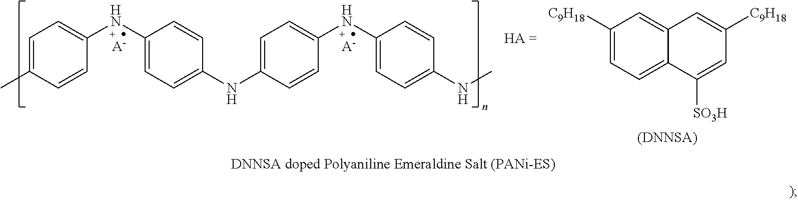

self-doped polyanilines (examples of self-doped polyanilines follows:

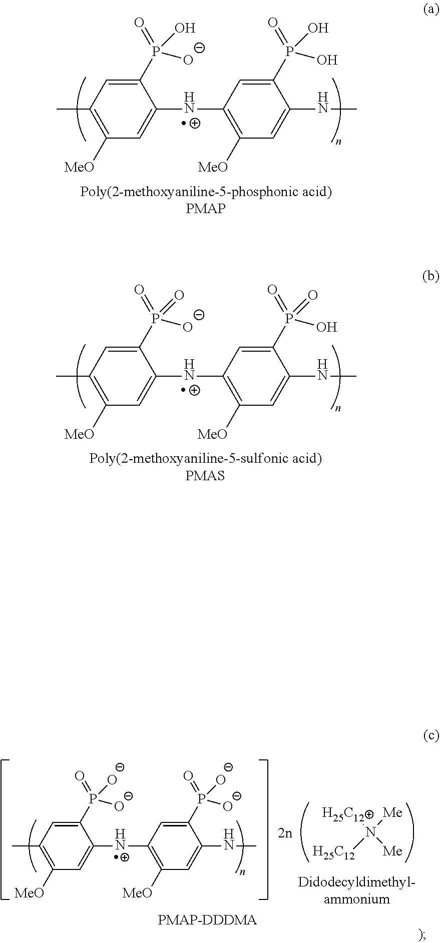

self-doped graft co-polymers (an example of self-doped graft co-polymers follows:

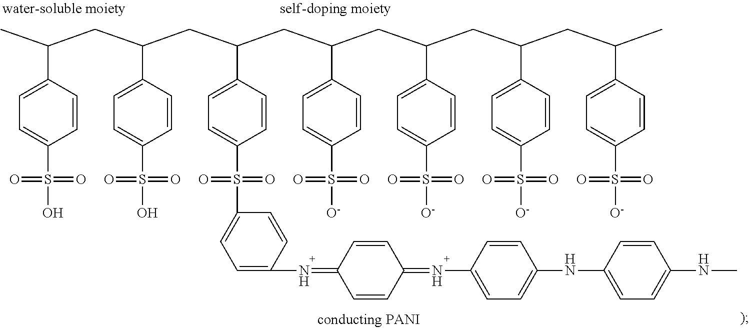 [SMILES: CNc1ccc(Nc2ccc(N=C3C=CC(=Nc4ccc(S(=O)(=O)c5ccc(C(CC(CC(C)c6ccc(S(=O)(=O)O)cc6)c6ccc(S(=O)(=O)O)cc6)CC(CC(CC(CC(C)c6ccc(S(=O)(=O)[O-])cc6)c6ccc(S(=O)(=O)[O-])cc6)c6ccc(S(=O)(=O)[O-])cc6)c6ccc(S(=O)(=O)[O-])cc6)cc5)cc4)C=C3)cc2)cc1]

and
polymer dopants (an example of a polymer dopant follows:

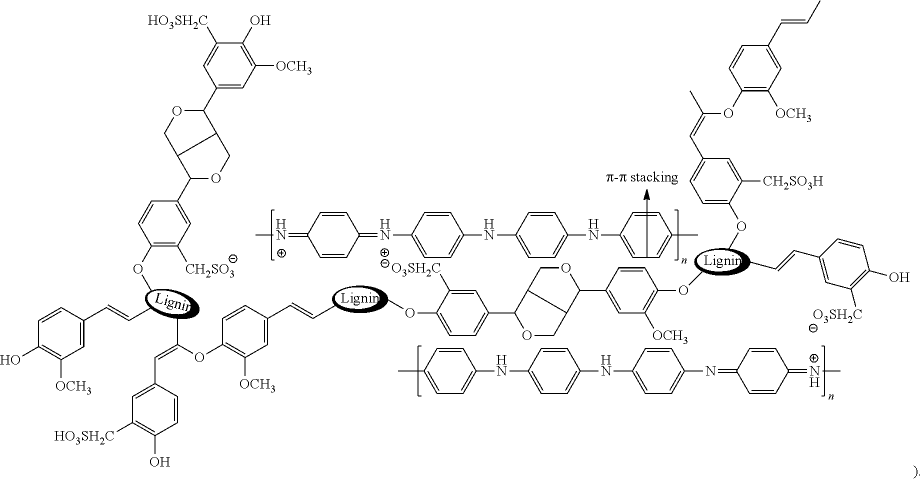

The individual organic semiconductor materials have various sizes. The individual organic semiconductor materials may be the same size, have one or more different sizes, or have all different sizes. For example, the size of at least a portion of or all of the distinct sensors in the array of sensors is 100 μm to 5 inches, including every integer μm value and range therebetween (e.g., 1 mm to 5 inches, 100 μm to 1 cm). The array of sensors may be 100 μm to 5 inches by 100 μm to 5 inches, including every integer μm value and range therebetween (e.g., 1 mm by 1 mm). The sensor element is a domain comprising a particular organic semiconducting material.

An array of sensors may be arranged as a planar array and/or a vertically stacked array. A planar array may be a plurality of sensors disposed a portion of a substrate. A vertically stacked array may be a plurality of sensors where a first sensor is disposed on at last a portion of or all of a substrate and subsequent sensors are disposed on the first sensor (e.g., the first sensor is disposed on a substrate, a second sensor is disposed on the first sensor, a third sensor is disposed on the second sensor, and so on or a first sensor is disposed on a substrate and a plurality of additional sensors are disposed on the first substrate, where additional sensors may be disposed on the plurality of additional sensors or a combination thereof). A plurality of sensors may be provided as a stack, where, for example, the sensors are disposed upon each other. An array of sensors may comprise more than one sensor stacks and/or additional sensors arranged in a planar orientation. Each sensor in a stack may be a distinct layer of an organic semiconducting material. One or more sensors in an array (e.g., a planar and/or vertically stacked array) may have the same or different thickness.

The individual organic semiconductor materials may be present as individual layers and have various thicknesses. The thickness is along a direction normal to the longest dimension or largest area of the organic semiconductor material layer. The individual organic semiconductor materials may have the same thickness, have one or more different thicknesses, or have all different thicknesses. For example, the thickness of at least a portion of or all of the distinct sensors in the sensor array is 1 μm to 2 mm, including every 0.1 μm value and range therebetween (e.g., 100 μm to 500 μm). The sensor element is a domain comprising a particular organic semiconducting material.

A sensor array may comprise various numbers of distinct sensor(s). In various examples, the number of distinct sensors in the sensor array is chosen from 2 to 1,000 (e.g., 2 to 10, 2 to 20, 5 to 20, 2 to 100, 5 to 100, or 2 to 500), including all integer numbers of distinct sensor(s) and ranges thereof therebetween.

A sensor may comprise an organic semiconducting material and an organic non-semiconducting material, which may be insulating materials. In various examples, one or more of the distinct sensors comprises one or more polymeric material(s) other than the organic semiconducting material. Non-limiting examples of polymeric materials, which are not organic semiconducting materials, include thermoplastic polymers, thermoset resins, elastomers, and the like, and combinations thereof. The organic semiconducting material(s) may be combined (e.g., blended) with one or more polymeric materials, which are not organic semiconducting materials, before or after doping the organic semiconducting material, in the case of organic semiconducting materials that are post-polymerization doped.

Various biogas samples may be used. A biogas sample may also referred to herein as biogas specimen. A biogas sample is obtained directly (e.g., in the case of a breath sample from an individual) or indirectly (e.g., the headspace gas from a liquid or tissue sample from an individual). Non-limiting examples of biogas samples include breath from an individual and/or a gas sample derived from (e.g., evolved from) from one or more bodily fluid(s) (such as, for example, sweat, urine, saliva, blood, and the like), cells, stool, tissue, cell culture, tissue culture, organ culture and the like, and combinations thereof. The biogas sample may contain vapor materials such as water vapors and aqueous aerosols.

The biogas sample may be obtained from the headspace of a sample, which may be a heated sample (e.g., a liquid sample or a solid sample) from an individual. In various examples, the heated sample is blood, tissue, cells, saliva, stool, or the like, or a combination thereof. The samples may be obtained from an individual. The individual may be a human or a non-human animal.

In the case of an organic semiconducting material that is protonated by one or more component of the test biogas sample, the test biogas sample comprises one or more acidic component that can protonate a basic site of the organic semiconducting material.

A method may further comprise recording a response from a sensor array. The response may be linear or non-linear. In various examples, the response is an electronic response (e.g., a change in impedance, resistivity, or the like, or a combination thereof). In various examples, the recording is carried out using a voltage measuring device, a current measuring device, an impedance measuring device, a transistor drain current of a threshold voltage measuring device, an imaging CMOS spectrometer, or the like, or a combination thereof. In various other examples, the response is a spectrophotometric response (e.g., a change in one or more wavelengths and/or intensity of the electronic response of the organic semiconducting material).

A method may further comprise generating a pattern based on the response of the plurality of distinct sensors; and comparing the pattern obtained from the test biogas sample to a pattern obtained from a reference biogas sample (e.g., control biogas sample) that corresponds to a particular physiological state or disease state to determine the presence or absence of a physiological state and/or diseased state.

Depending upon the gas profile of the biogas sample, a distinct pattern will be generated based on the responses of the sensors. The response of each sensor is characterized as whether a sensor is responding or not, and/or other characteristics of the response (such as the intensity of the response). The pattern may then be compared to a control pattern. In certain examples, the specific VOCs and/or amounts of specific VOCs are not determined.

The control pattern may be a positive control pattern or a negative control pattern. A negative control pattern can be generated by a gas specimen which has an ensemble of gaseous components known to be associated with the absence of a particular physiological state and/or disease state. A positive control pattern, on the other hand, can be generated by gas specimen which has an ensemble of gaseous components known to be associated with the presence of a particular physiological state and/or disease state.

Another method according to the present disclosure utilizes pattern-matching techniques in the readout and analysis component. In this method, a pattern may be generated based on the response of the sensor array. The pattern can be, for example, a two-dimensional array of values corresponding to the sensor array, a three-dimensional array of values corresponding to the sensor array, a histogram, or the like. The pattern can be compared to the pattern of a control gas specimen, or a combination of several control gas specimens, to determine the presence or absence of a physiological state and/or diseased state.

The comparing may be carried out in various ways. For example, in the case of patterned based responses, the comparing is carried out by visual inspection.

In another example, the comparing comprises subjecting the response of the plurality of distinct sensors to a predetermined rule set, where the predetermined rule set defines a particular physiological state and/or diseased state, thereby identifying the presence or absence of a particular physiological state and/or diseased state. The predetermined rule set may define the predetermined control gas, thereby enabling matching of the test gas sample with the predetermined control gas sample. In various other examples, the comparing comprises principal component analysis, vector analysis, fuzzy logic, Monte Carlo analysis, or the like, or a combination thereof.

The presence of (and in certain cases, the amount of) and/or absence of VOCs in a biogas sample may distinguish between patients with non-healthy patients (e.g., a cancer patient) and healthy patients. Non-limiting examples of volatile organic compounds that can be detected using methods of the present disclosure include, 1-methyl-4-(1-methyl)benzene; toluene; dodecane; 3,3-dimethyl pentane; 2,3,4-trimethyl hexane; 1,1′-(1-butenylidene)bis benzene; 1,3-dimethyl benzene; 1-iodo nonane; (1,1-dimethylethyl thio) acetic acid; 4-(4-propylcyclohexyl)-4′-cyano [1,1′-biphenyl]4-yl ester benzoic acid; 2 amino-5-isopropyl-8-methyl-1-azulenecarbonitrile; 5-(2-methylpropyl) nonane; 2,3,4-trimethyl decane; 6-ethyl-3-octyl ester 2 trifluormethyl benzoic acid; p-xylene; and 2,2-dimethyldecane (the presence or absence of which may distinguish between patients with cancer and healthy patients); butane; 3-methyl tridecane; 7-methyl tridecane; 4-methyl octane; 3-methyl hexane; heptane; 2-methyl hexane pentane; 5-methyl decane, isobutane; methanol; ethanol; acetone; pentane; isoprene; isopropanol; dimethylsulfide; carbon disulfide; benzene; toluene, styrene; decane; isoprene; benzene; undecane; 1-hexene; propyl benzene; 1,2,4-trimethyl benzene; heptanal; methyl cyclopentane (the presence or absence of which may distinguish between patients with cancer and healthy patients); pentane, 3-methylhexane, decene, caryophyllene, naphthalene, trichloroethylene, heptanal, acetophenone, isopropyl myristate and 2-propanol (the presence or absence of which may distinguish between patients with breast cancer and healthy patients); and 4 methyl octane, 2,4-dimethyl heptane, isopropanol, toluene, isoprene, alkane, acetic acid, acetone, 2,6 I I-trimethyl dodecane, 3,7-dimethyl undecane, and 2,3-dimethyl heptane (the presence or absence of which may distinguish between patients with asthma and healthy patients).

The comparing may identify the presence of one or more classes of compounds and/or specific compounds (e.g., one or more classes of and/or specific organic compounds) in the biogas sample and the presence of the one or more specific compounds determine the presence or absence of a physiological state and/or diseased state. The classes of one or more compounds may be physiologically/biologically-relevant organic compounds (e.g., volatile organic compounds), reactive oxygen species, physiologically/biologically-relevant gases (e.g., NO, CO, CO₂, H₂O, NH₃, alkanes of various carbon lengths, oxidized alkanes, alcohols, aldehydes, ketones, carboxylic acids, esters, aromatic compounds (such as, for example, benzene and the like), modified aromatic compounds (such as, for example, toluene sulfonic acid, benzyl alcohol, and the like), sulfides (such as, for example, dimethyl sulfide), amines, and the like), and the like, and combinations thereof. The compounds may be volatile compounds. Classes of compounds may refer to compounds with structural and/or reactive similarity (e.g., ketones, aldehydes, aromatics/aryls, esters, and the like).

The comparing may identify the concentration of one or more of the one or more classes of compounds and/or specific compounds (e.g., one or more classes of and/or specific organic compounds) in the biogas sample and the presence and concentration of the one or more specific compounds determine the presence or absence of a physiological state and/or diseased state. In various examples, the number of classes of compounds and/or specific compounds is 1 to 100, including all integer values and ranges therebetween.

In various examples, a method is based on multispectral analysis. Vector analysis may be used for the comparison of the matrices of data collected from the sensor(s). The extent of the presentation is controlled with end markers determining the quantification of the data analysis obtained with different sensors. Some sensors can still not differentiate certain scents but another sensor may be able to separate at least one pair of scents resulting in the specification of all scents inside the map expressed by the end markers.

In various other examples, a method is based on machine learning. In various other examples, a method is based on use of neural networks.

The methods can be used to determine the presence or absence of various physiological and/or disease states of an individual. The present method can be used to detect physiological and/or diseased states by, for example, comparing a specific pattern obtained from a test biogas specimen to predetermined controls.

In various examples, disease state is chosen from cancer (e.g., lung cancer, breast cancer, ovarian cancer, and the like), diabetes, autoimmune diseases (such as, for example, HIV/AIDS and the like), mental illness (such as, for example, schizophrenia and the like), metabolic diseases, and the like, and combinations thereof.

These methods may be carried out at early stages where symptoms might not be visible and this allows earlier diagnoses on patients. The earlier the diagnosis, the more possibility there is to treat the cancer and save the patient.

The methods can be used in combination with other treatment modalities. In various examples, one or more of the method(s) is used pre-surgery or post-surgery and/or in combination with one or more conventional therapy (e.g., chemotherapy, and the like).

Various physiological states can be determined. A physiological stage may be a natural physiological state or an altered physiological state. A non-limiting example of a natural physiological state is pregnancy. An altered physiological state may be exogenously induced. An altered physiological stage may result from an external stimulus or stimuli (such as, for example, physical stimuli, chemical stimuli (e.g., drugs, which may be pharmaceutical agents, drugs of abuse, alcohol, and the like, and combinations thereof), and the like, and combinations thereof). The methods may be diagnostic methods, drug screening methods, drug efficacy methods, and the like.

The present method can also be used for detection of non-physiological states by evaluation of gas samples other than biogas samples. For examples, gas samples such as environmental samples, samples from chemical plants or processes or the like can be used. For example, a method comprises matching the response for a test gas sample to a predetermined reference gas sample (e.g., control gas sample). The method comprises providing a sensor array comprising a plurality of distinct sensors as described herein. The array is exposed to the test gas sample and the responses of a plurality of distinct sensors are recorded. The test gas sample and the predetermined reference gas sample (e.g., control gas samples) responses are then compared to evaluate if the two are matching. The evaluation can be carried out, for example, by visual inspection of the patterns generated by the sensors or by other comparison modes described herein.

In an aspect, the present disclosure provides sensors. A sensor may comprise one or more layer(s) of organic semiconducting material. The sensors may be used in a method of the present disclosure or a system of the present disclosure.

In various examples, a sensor comprises one or more layer of organic semiconducting material, and the individual layers may be a layer of organic semiconducting material which may be i) at least partially oxidized or ii) at least partially doped or iii) is protonated by one or more component of the test biogas sample. In various examples, the organic semiconducting material (described herein) is independently for each sensor chosen from organic semiconducting oligomers and organic semiconducting polymers, and combinations thereof, and the like. The sensor materials can have various sizes (e.g., various areas and/or thicknesses).

The individual sensors may also be configured to be in electrical communication with other components (e.g., a recording device, an analysis device, a processor, a network device, a transmitter, and the like, and combinations thereof). For example, the sensor comprises one or more of electrical contacts, terminals, leads, ports, and the like.

In an aspect, the present disclosure provides systems. The systems can be used for detecting physiological states and/or disease states. In various examples, a system is used to carry out a method of the present disclosure. Non-limiting examples of systems of the present disclosure are provided in the draft sample claims.

The system (e.g., system comprising a sensor array) may be provided in the form of a device. The identification of the physiological and/or diseased state may be achieved without the necessity to identify the individual components of the biogas sample. The altered electrical signals from the sensors (which encodes information about the presence of the components of the biogas sample and their respective concentrations) are communicated to other components to be processed further. The readout and analysis component may be software that interprets the signals and correlates them to a particular physiological and/or disease state.

In various examples, a system comprises: optionally, a vessel for heating a liquid, the vessel having a vapor outlet; optionally, a channel connected to the vapor outlet for receiving evaporate from the vessel; a sensor array having one or more sensor(s) (e.g., a plurality of sensors), where the sensor(s) may be configured to contact evaporate in the channel, and where each sensor of the one or more sensor(s) comprises: a first electrical contact; a second electrical contact; and a layer of organic semiconducting material connecting the first electrical contact to the second electrical contact.

A system may further comprise a source of gas. The source of gas may be configured to bring at least a portion of the biogas into contact with the sensor array.

The sensors may be oriented with respect to each other in various ways. In various examples, the sensor array comprises a planar array and/or vertically stacked array. In a system or a sensor with a plurality of individual organic semiconducting material layers, individual layers of organic semiconducting materials can be oriented in various ways. Individual layers may be coplanar or vertically stacked.

A system may further comprise a recording and/or analysis device. Each such device may be in electrical communication with the sensor array (e.g., in electrical contact with each sensor of the array.

The steps of the methods described in the various embodiments and examples disclosed herein are sufficient to carry out the methods of the present disclosure. Thus, in an various examples, a method consists essentially of a combination of the steps of the methods disclosed herein. In various other examples, a method consists of such steps.

The following Statements describe various embodiments of the present disclosure.

Statement 1. A method for diagnosing a physiological state and/or diseased state by analysis of a test biogas sample comprising providing a sensor array comprising a plurality of distinct sensor(s), where each distinct sensor comprises a layer of organic semiconducting material which may be i) at least partially oxidized or ii) at least partially doped or iii) is protonated by one or more component of the test biogas sample, where the each individual distinct sensor differs in terms of one or more or all of dopant, dopant concentration, or organic semiconducting material from other distinct sensors; and exposing the sensor array to the test biogas sample, where the exposing generates a response from the sensor array, where the response correlates to the presence or absence of a physiological state and/or diseased state. In the case of an organic semiconducting material that is protonated by one or more component of the test biogas sample, the test biogas sample comprises one or more acidic component that can protonate a basic site of the organic semiconducting material.

Statement 2. A method according to Statement 1, where the organic semiconducting material is independently for each sensor chosen from organic semiconducting oligomers and organic semiconducting polymers, and combination thereof, and the like. Non-limiting examples of organic semiconducting polymers include polyaniline (PANI), polythiophene, polypyrrole, and substituted analogs thereof, and the like.

Statement 3. A method according to Statements 1 or 2, where the organic semiconducting material is independently for each sensor chosen from polyaniline (PANI), polythiophene, polypyrrole, substituted analogs of any of the foregoing, block copolymers comprising one or more block thereof, graft copolymers comprising one or more block thereof, network polymers thereof, and combinations thereof, and the like.

The organic semiconducting material may be formed by oxidative polymerization of monomers, such as, for example, aniline, thiophene, pyrrole, substituted analogs of any of the foregoing (e.g., carboxylated, sulfonated, phosphonylated, alkylated, alkoxylated, etherified analogs thereof, or the like, or a combination thereof), and the like, and combinations thereof. The copolymers and network copolymers may comprise polymerized monomers, such as, for example, non-electroactive monomers with electroactive monomers. Non-limiting examples of non-electroactive monomers include all vinyl linked monomers, difunctional esters, alcohols, carboxylic acids, and the like.

Statement 4. A method according to any one of the preceding Statements, where the one or more of the organic semiconducting material(s) is a polymer/are polymers and the polymer(s) is/are modified post polymerization (e.g., sulfonated, anion exchanged, cation exchanged, or the like, or a combination thereof).

Statement 5. A method according to any one of the preceding Statements, where the organic oligomeric semiconducting material has a molecular weight (Mw and/or Mn, which may be determined by methods known in the art, such as, for example, by size exclusion chromatography, which may be carried out by comparison to one or more polystyrene standard(s)) of 10-500 g/mol, including all 0.1 g/mol values and ranges therebetween, and/or the organic polymeric semiconducting material with Mw has a molecular weight (Mw and/or Mn, which may be determined by methods known in the art, such as, for example, by size exclusion chromatography, which may be carried out by comparison to one or more polystyrene standard(s)) of 200-500,000 g/mol, including all 0.1 g/mol values and ranges therebetween.

Statement 6. A method according to any one of the preceding Statements, where the individual organic semiconducting material(s) is/are amorphous or at least partially crystalline or amorphous.

Statement 7. A method according to any one of the preceding Statements, where the dopant is independently for each sensor chosen from oxidants, acids (e.g., mineral acids (such as, for example, hydrochloric acid, sulfonic acid, nitric acid, and the like, organic acids (such as, for example, sulfonic acids (e.g., camphor sulfonic acid, p-toluene sulfonic acid, alkylated (e.g., C₁-C₂₀ alkyl), aryl (e.g., benzene, naphthalene, and the like), phosphonic acids, and the like), polymers (such as, for example, sulfonated polystyrene, polyphosphazines, self-doped sulfonated polyanilines, and the like), and the like, and combinations thereof. It may be desirable to use one or more oxidant(s) as dopants for polythiophenes and polypyrroles.

Statement 8. A method according to Statement 7, where the organic semiconducting material is a self-doped organic conducing material (e.g., poly(2-methoxyaniline-5-phosphonic acid) (PMAP), poly(2-methoxyaniline-5-sulfonic acid, and the like), and copolymers (e.g., bock copolymers, graft copolymers, and the like), network polymers thereof.

Statement 9. A method according to any one of the preceding Statements, where the dopant concentration is independently for each sensor 2 to 50 weight % (based on the total weight of the organic semiconducting material and dopant(s)), including all 0.1 weight % values and Statement 10. A method according to any one of the preceding Statements, where the size of at least a portion of or all of the distinct sensors in the sensor array is 100 μm to 5 inches, including every integer μm value and range therebetween (e.g., 1 mm to 5 inches, 100 μm to 1 cm). The sensor array may be 100 μm to 5 inches by 100 μm to 5 inches, including every integer μm value and range therebetween (e.g., 1 mm by 1 mm).

Statement 11. A method according to any one of the preceding Statements, where the number of distinct sensors in the sensor array is chosen from 2 to 1,000 (e.g., 2 to 10, 2 to 20, 5 to 20, 2 to 100, 5 to 100, and 2 to 500), including all integer numbers of distinct sensors and ranges thereof therebetween.

Statement 12. A method according to any one of the preceding Statements, where one or more of the distinct sensors further comprises one of more polymeric material other than the organic semiconducting material. Non-limiting examples of polymeric materials include thermoplastic polymers, thermoset resins, elastomers, and the like, and combinations thereof.

Statement 13. A method according to any one of the preceding Statements, where the biogas sample is breath and/or a gas sample derived from (e.g., evolved from) from one or more bodily fluid(s) (such as, for example, sweat, urine, saliva, blood, and the like), cells, stool, tissue, and the like, and combinations thereof.

Statement 14. A method according to any one of the preceding Statements, where the biogas sample is obtained from the headspace of a heated sample (e.g., a liquid sample or a solid sample) from an individual.

Statement 15. A method according to Statement 14, where the heated sample is blood, tissue, cells, saliva, stool, or the like, or a combination thereof.

Statement 16. A method according to any one of the preceding Statements, further comprising recording the response from the sensor array. E.g., the recording is carried out using a voltage measuring device, a current measuring device, an impedance measuring device, a transistor drain current of a threshold voltage measuring device, an imaging CMOS spectrometer, or the like, or a combination thereof.

Statement 17. A method according to any one of the preceding Statements, where the response is a spectrophotometric response (e.g., a change in one or more wavelengths and/or intensity of the electronic response of the organic semiconducting material). The response may be linear or non-linear.

Statement 18. A method according to any one of Statements 1-16, where the response is an electronic response (e.g., a change in impedance, resistivity, or the like, or a combination thereof).

Statement 19. A method according to any of the preceding Statements, where the method further comprises generating a pattern based on the response of the plurality of distinct sensors; and comparing the pattern obtained from the test biogas sample to a pattern obtained from a control biogas sample (e.g., reference biogas sample) to determine the presence or absence of a physiological state and/or diseased state.

Statement 20. A method according to Statement 19, where, in the case of patterned based responses, the comparing is carried out by visual inspection.

Statement 21. A method according to Statement 19, where the comparing comprises subjecting the response of the plurality of distinct sensors to a predetermined rule set, where the predetermined rule set defines a particular physiological state and/or diseased state, thereby identifying the presence or absence of a particular physiological state and/or diseased state.

Statement 22. A method according to Statement 21, where the predetermined rule set defines the predetermined control gas, thereby enabling matching of the test gas sample with the predetermined control gas sample.

Statement 23. A method according to Statement 19, where the comparing comprises principal component analysis, vector analysis, fuzzy logic, Monte Carlo analysis, or the like, or a combination thereof.

Statement 24. A method according to Statement 19, where the comparing identifies the presence of one or more classes of compounds and/or specific compounds (e.g., one or more classes of and/or specific organic compounds) in the biogas sample and the presence of the one or more specific compounds determine the presence or absence of a physiological state and/or diseased state.

Statement 25. A method according to Statement 24, where the classes of one or more compounds are physiologically/biologically-relevant organic compounds (e.g., volatile organic compounds), reactive oxygen species, physiologically/biologically-relevant gases (e.g., NO, CO, CO₂, H₂O, NH₃, alkanes of various carbon lengths, oxidized alkanes, alcohols, aldehydes, ketones, carboxylic acids, esters, aromatic compounds (such as, for example, benzene and the like), modified aromatic compounds (such as, for example, toluene sulfonic acid, benzyl alcohol, and the like), sulfides (such as, for example, dimethyl sulfide), amines, and the like), and the like, and combinations thereof.

Statement 26. A method according to any one of Statements 19-25, where the comparing identifies the concentration of one or more of the one or more classes of compounds and/or specific compounds (e.g., one or more classes of and/or specific organic compounds) in the biogas sample and the presence and concentration of the one or more specific compounds determine the presence or absence of a physiological state and/or diseased state.

Statement 27. A method according to any one of Statements 24-26, where the number of classes of compounds and/or specific compounds is 1 to 1,500, including all integer values and ranges therebetween (e.g., 1-1,000 or 1-500).

Statement 28. A method according to any one of the preceding Statements, where the disease state is chosen from cancer (e.g., lung cancer, breast cancer, ovarian cancer, and the like), diabetes, autoimmune diseases (such as, for example, HIV/AIDS and the like), mental illness (such as, for example, schizophrenia and the like), metabolic diseases, and the like, and combinations thereof and/or the physiological state is a natural physiological state or an altered physiological state. A non-limiting example of a natural physiological state is pregnancy. An altered physiological state may be exogenously induced. An altered physiological stage may result from an external stimulus or stimuli (such as, for example, physical stimuli, chemical stimuli (e.g., drugs, which may be pharmaceutical agents, drugs of abuse, alcohol, and the like, and combinations thereof), and the like, and combinations thereof).

Statement 29. A method according to any one of the preceding Statements, where the method is used pre-surgery or post-surgery and/or in combination with one or more conventional therapy (e.g., chemotherapy, and the like).

Statement 30. A sensor comprising one or more layer of organic semiconducting material, and the individual layers may be a layer of organic semiconducting material which may be i) at least partially oxidized or ii) at least partially doped or iii) is protonated by one or more component of the test biogas sample.

Statement 31. A sensor according to Statement 30, where the organic semiconducting material is independently for each sensor chosen from organic semiconducting oligomers and organic semiconducting polymers, and combinations thereof, and the like.

Statement 32. A sensor according to Statements 31 or 32, where the organic semiconducting material is independently for each sensor chosen from polyaniline (PANI), polythiophene, polypyrrole, and substituted analogs thereof, and the like, block copolymers comprising one or more block thereof, graft copolymers comprising one or more block thereof, network polymers thereof, and combinations thereof, and the like.

Statement 33. A sensor according to any one of Statements 30-32, where the one or more of the organic semiconducting material(s) is a polymer/are polymers and the polymer(s) is/are modified post polymerization (e.g., sulfonated, anion exchanged, cation exchanged, or the like, or a combination thereof).

Statement 34. A sensor according to any one of Statements 30-33, where the organic oligomeric semiconducting material has a molecular weight (Mw and/or Mn, which may be determined by methods known in the art, such as, for example, by size exclusion chromatography, which may be carried out by comparison to one or more polystyrene standard(s)) of 10-500 g/mol, including all 0.1 g/mol values and ranges therebetween, and/or the organic polymeric semiconducting material with Mw has a molecular weight (Mw and/or Mn, which may be determined by methods known in the art, such as, for example, by size exclusion chromatography, which may be carried out by comparison to one or more polystyrene standard(s)) of 200-500,000 g/mol, including all 0.1 g/mol values and ranges therebetween.

Statement 35. A sensor according to any one of Statements 30-34, where the individual organic semiconducting material(s) is/are amorphous or at least partially crystalline or amorphous.

Statement 36. A sensor according to any one of Statements 30-35, where the dopant is independently for each sensor chosen from oxidants, acids (e.g., mineral acids (such as, for example, hydrochloric acid, sulfonic acid, nitric acid, and the like, organic acids (such as, for example, sulfonic acids (e.g., camphor sulfonic acid, p-toluene sulfonic acid, alkylated (e.g., C₁-C₂₀ alkyl) aryl (e.g., benzene, naphthalene, and the like), phosphonic acids, and the like), polymers (such as, for example, sulfonated polystyrene, polyphosphazines, self-doped sulfnoated polyanilines, and the like), and the like, and combinations thereof.

Statement 37. A sensor according to any one of Statements 30-36, where the organic semiconducting material is a self-doped organic conducing material (e.g., poly(2-methoxyaniline-5-phosphonic acid) (PMAP), poly(2-methoxyaniline-5-sulfonic acid), and the like, and copolymers (e.g., bock copolymers, graft copolymers, and the like) and network polymers thereof.

Statement 38. A sensor according to any one of Statements 30-37, where the dopant concentration is independently for each sensor 2 to 50 weight % (based on the total weight of the organic semiconducting material and dopant(s)), including all 0.1 weight % values and ranges therebetween.

Statement 39. A sensor according to any one of Statements 30-38, where the size of at least a portion of or all of the distinct sensors in the sensor array is 100 μm to 5 inches, including every integer μm value and range therebetween (e.g., 1 mm to 5 inches, 100 μm to 1 cm). The sensor array may be 100 μm to 5 inches by 100 μm to 5 inches, including every integer μm value and range therebetween (e.g., 1 mm by 1 mm).

Statement 40. A system comprising: a vessel for volatilizing (e.g., heating) a liquid, the vessel having a vapor outlet; a channel connected (e.g., in liquid and/or gas communication) to the vapor outlet for receiving evaporate from the vessel; a sensor array having at least one sensor (e.g., one or more sensor according to any one of Statements 30-39), where the at least one sensor is configured to contact evaporate in the channel, and where each sensor of the at least one sensor comprises: a first electrical contact; a second electrical contact; and a layer of organic semiconducting material connecting the first electrical contact to the second electrical contact, which may be i) at least partially oxidized or ii) doped, where each individual distinct sensor differs in terms of one or more or all of dopant, dopant concentration, or organic semiconducting material from other distinct sensors.

Statement 41. A system according to Statement 40, further comprising a source of a gas, configured to bring at least a portion of the biogas into contact with the sensor array.

Statement 42. A system according to Statements 40 or 41, where the sensor array comprises a planar array and/or vertically stacked array.

Statement 43. A system according to any one of Statements 40-42, where the system further comprises a recording and/or analysis device in in electrical communication with the sensor array.

Statement 44. A method for identifying the presence or absence of a physiological state and/or a disease state by analysis of a test biogas sample from an individual or a biological sample comprising: providing an array of sensors where each sensor of the array comprises an organic semiconducting material, where at least two sensors of the array have different organic semiconducting material or have the same semiconducting material at different concentrations, and the oxidation state of the organic semiconducting material of each sensor changes in response to one or more specific volatile compounds, and the at least two sensors can detect distinct volatile compounds or the same volatile compound at different concentrations; and contacting the array of sensors with the test biogas sample, where if the one or more specific volatile compounds are present, the distinct sensors reactive to the specific volatile compounds or a different concentration of the same compound exhibit separately detectable responses and based on the combination of detected responses, the presence or absence of the physiological state and/or disease state is identified.

Statement 45. A method according to Statement 44, where the organic semiconducting material is independently for each sensor chosen from organic semiconducting oligomers, organic semiconducting polymers, and combinations thereof, and, optionally, when the organic semiconducting material is an organic semiconducting polymer, the organic semiconducting polymer is modified post polymerization.

Statement 46. A method according to Statement 45, where the organic semiconducting material is independently for each sensor chosen from polyaniline (PANI), polythiophene, polypyrrole, substituted analogs thereof, block copolymers comprising one or more block thereof, graft copolymers comprising one or more block thereof, network polymers thereof, and combinations thereof.

Statement 47. A method according to Statements 45 and 46, where the organic semiconducting oligomers have a molecular weight (M_{w} and/or Mₙ) of 10-500 g/mol, and/or the organic semiconducting polymers have a molecular weight (M_{w} and/or Mₙ) of 200-500,000 g/mol.

Statement 48. A method according to any one of Statements 44-47, where the individual organic semiconducting material(s) is/are amorphous or at least partially crystalline or amorphous.

Statement 49. A method according to any one of Statements 44-48, where the organic semiconducting material(s) further comprise a dopant and the dopant is independently for each sensor chosen from oxidants, acids, polymers, and combinations thereof, or the organic semiconducting material is a self-doped organic conducting material, copolymers thereof, or network polymers thereof.

Statement 50. A method according to Statement 49, where the dopant concentration is independently for each sensor 2 to 50 weight % (based on the total weight of the organic semiconducting material and dopant(s)).

Statement 51. A method according to any one of Statements 44-50, where the array of sensors is a plurality of sensors and the plurality of sensors is provided as a stack

Statement 52. A method according to Statement 51, where the array of sensors comprises one or more stacked sensors.

Statement 53. A method according to any one of Statements 44-52, where the array of sensors are arranged as a planar array and/or vertically stacked array.

Statement 54. A method according to any one of Statements 44-53, where the number of distinct sensors in the array of sensor is chosen from 2 to 1,000.

Statement 55. A method according to any one of Statements 44-54, where one or more of the distinct sensors further comprise one or more polymeric materials other than the organic semiconducting material(s).

Statement 56. A method according to any one of Statements 44-55, where the test biogas sample is breath and/or a gas sample derived from one or more bodily fluid(s), cells, stool, tissue, or a combination thereof.

Statement 57. A method according to any one of Statements 44-56, further comprising recording the response from the sensor array and generating a pattern based on the response of the plurality of distinct sensors; and comparing the pattern obtained from the test biogas sample to a pattern obtained from a reference biogas sample, which corresponds to a particular physiological or disease state to determine the presence or absence of the physiological state and/or disease state.

Statement 58. A method according to Statement 57, where the comparing comprises subjecting the response of the plurality of distinct sensors to a predetermined rule set, where the predetermined rule set defines the physiological state(s) and/or disease state(s), thereby identifying the presence or absence of particular physiological state(s) and/or disease state(s), and the comparing optionally comprises utilizing principal component analysis, vector analysis, fuzzy logic, Monte Carlo analysis, or a combination thereof.

Statement 59. A method according to any one of Statements 44-58, where the response is a spectrophotometric response or electronic response.

Statement 60. A method according to any one of Statements 44-59, where the disease state is chosen from cancers, diabetes, autoimmune diseases, mental illnesses, metabolic diseases, and combinations thereof and/or the physiological state is a natural physiological state or an altered physiological state.

Statement 61. A system comprising: a vessel for volatilizing (e.g., heating) a liquid, the vessel having a vapor outlet; a channel in communication with the vapor outlet for receiving evaporate from the vessel; an array of sensors having at least one sensor, arranged as a planar array and/or vertically stacked array, where the at least one sensor is configured to contact evaporate in the channel, and where each sensor of the at least one sensor comprises: a first electrical contact; a second electrical contact; and the sensors of the array of sensors comprise an organic semiconducting material, where at least two sensors have different organic semiconducting material or have the same semiconducting material at different concentrations, and the oxidation state of the organic semiconducting material of each sensor changes in response to one or more specific volatile compounds, and at least two sensors of the array of sensors can detect distinct volatile compounds or the same volatile compound at different concentrations. The system may be used to perform a method of according to the present disclosure (e.g., any one of Statements 44-60).

The following examples are presented to illustrate the present disclosure. They are not intended to be limiting in any matter.

### Example 1

This example provides a description of methods and system of the present disclosure.

Materials. Aniline (Sigma>99%) was freshly double distilled before use. Hydrochloric acid HCl (ACS reagent), ammonium persulfate (APS), ammonium hydroxide (25%), chloroform (>99%) and camphor sulfonic acid (CSA) were purchased from Sigma and were used as received. Thin Film film interdigitated platinum film electrodes (IDA) (line spacing 100 μm) were purchased from the Electronic Design Center, Case Western University, precision gas diluter Model 1010 was purchased from Custom Sensor Solutions. A 25 ppm ammonia/dry N₂ was used as the calibration standard and was purchased from Prest-O sales.

Experimental. Polyaniline (PANI) doped with HCl was prepared by chemical oxidative polymerization of aniline in aqueous acidic medium (1 M HCl) with APS as an oxidant. It has been reported earlier that higher polymerization yields were obtained by using oxidant to monomer ratio of 1.2. 50 ml of 0.48 M APS in 1 M HCl was added slowly to 50 ml, 0.4 M aniline solution in a beaker. The mixture was left to polymerize overnight at room temperature. The PANI precipitate was collected on a filter paper and washed repeatedly with 0.1 M HCl followed by repeated washes with acetone. Deprotonation of the resulting PANI salt was performed by stirring the powder in an aqueous 0.1 M NH₄OH solution for 24 hours at room temperature thus obtaining the emeraldine base (EB) which was then washed with water repeatedly until neutral pH and then dried under vacuum for 48 hours at 60° C. PANI was redoped with CSA with molar ratio of 1:2. A 0.5 wt % solution of the resulting PANI-CSA complex (37.5 mg PANI, 48 mg CSA) in 5 ml chloroform was prepared and allowed to dissolve for 2 days with constant stirring. The solutions were filtered with a 0.2 μm PTFE syringe filter to remove any particulate impurities.

An Agilent 4155C semiconductor analyzer was used for taking the measurements and customized test software was developed using the EasyDesktop software provided with the analyzer. Tedlar bags (5 L, prest-O sales and 0.5 L Zefon) were used to make the required dilution with the gas diluter. A DropSens flow cell setup was employed to inject ammonia gas over the electrode. Teflon tubing was used to connect the gas to the flow setup to minimize any NH₃ absorption. Measurements were made by applying a fixed potential of 1V to the sensor and measuring the resulting current, which changed as a function of gas passed over the sensor surface.

Sensor Fabrication. The films were prepared by spin coating the PANI-CSA solution on the IDA electrodes. Prior to spin coating, the electrodes were cleaned by rinsing in methanol followed by rinsing with deionized water and drying in a stream of dry nitrogen. PANI films were spun cast onto the IDA electrodes by adding 100 μl solutions at 500 RPM. The electrode pads were cleaned with a Q-tip dipped in methanol to facilitate direct electrical contact with the analyzer.

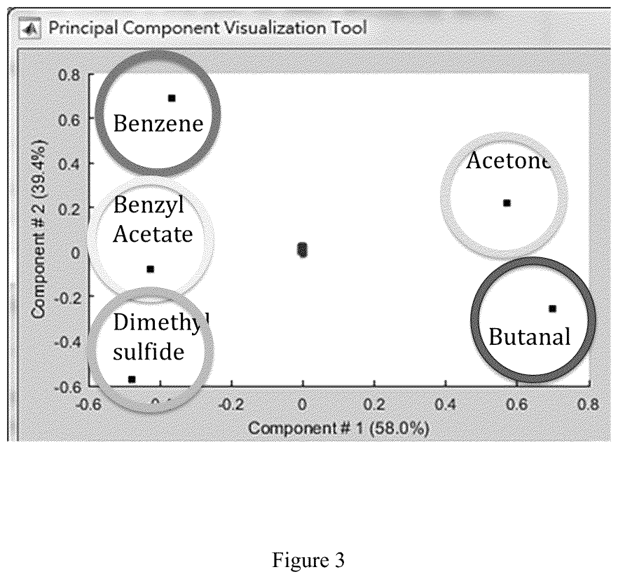

Framing the scents. Olfactory system is able to differentiate the scents based on their chemical characteristics so five examples were selected, their resistivities were measured with five different sensors (dopants for polyaniline). Here then we measured five different scents: Benzene as the aromatic, Benzyl acetate as the aromatic ester, dimethylsulfide as a sulfur compound, acetone as the ketone and butanal as the aldehyde. See FIG. 3.

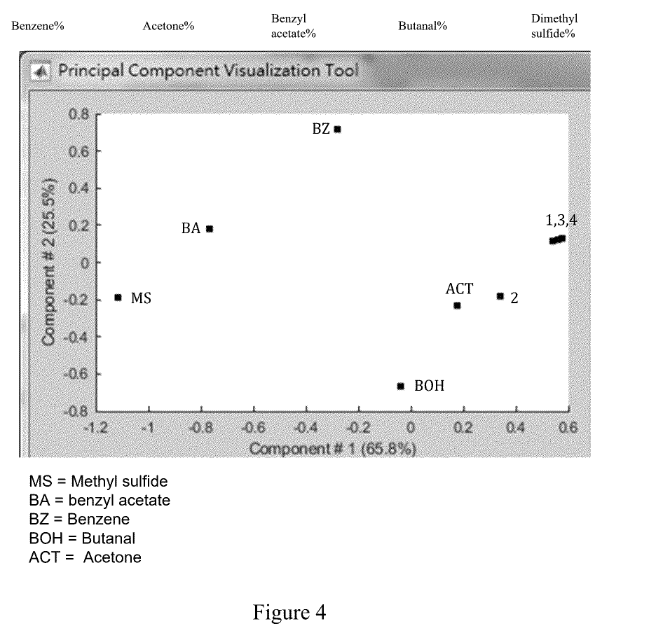

Four different wines were analyzed. See FIG. 4.

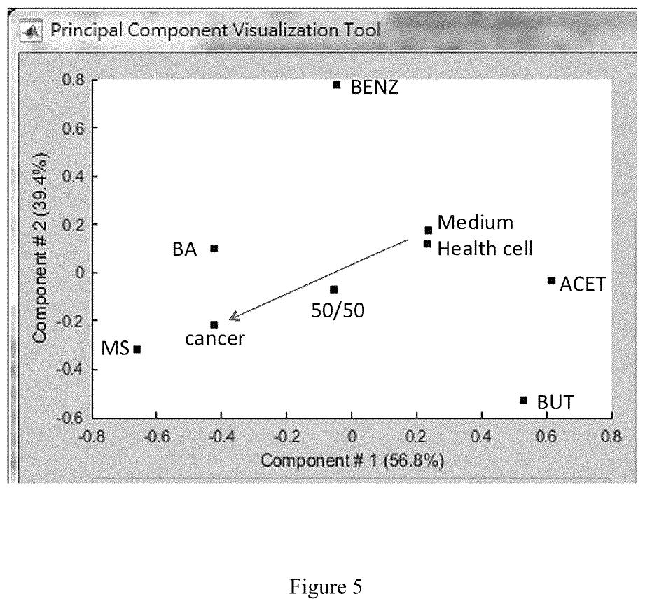

A sample was analyzed. Five different scents (extremes) were differentiated with the five different sensors. First the smell of medium only, then from healthy cells. Then cancer cells were added such that there was a 50:50 composition and eventually only cancer cells. See FIG. 5.

### Example 2

This example provides a description of methods and system of the present disclosure.

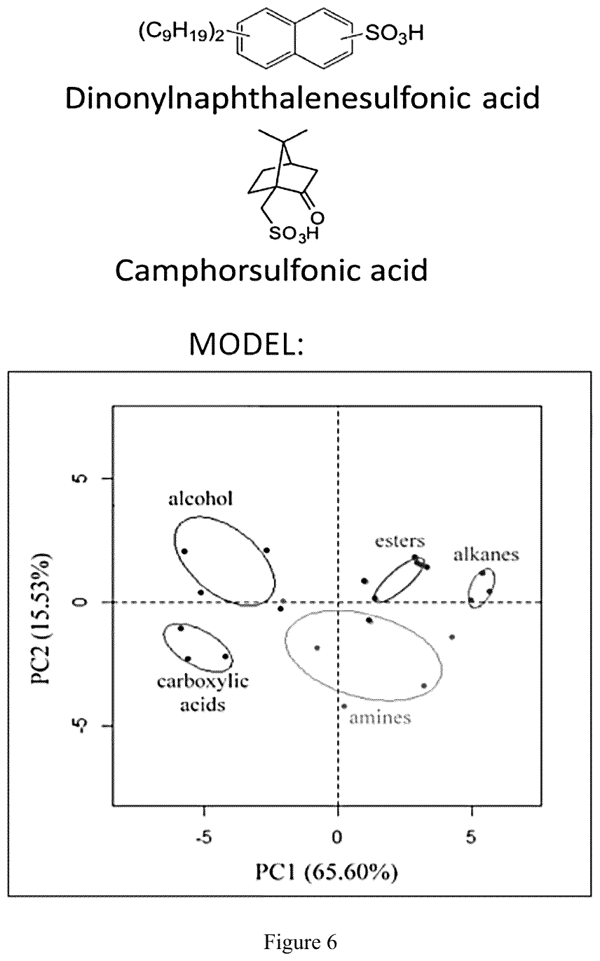

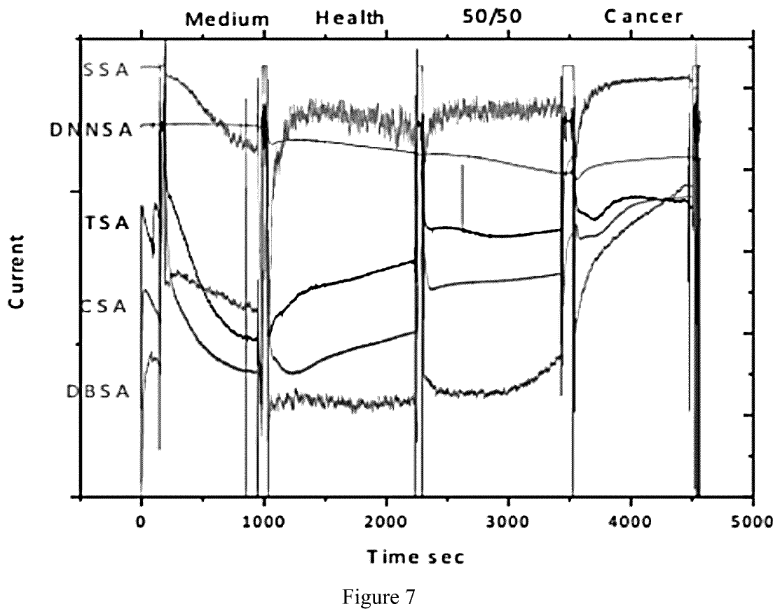

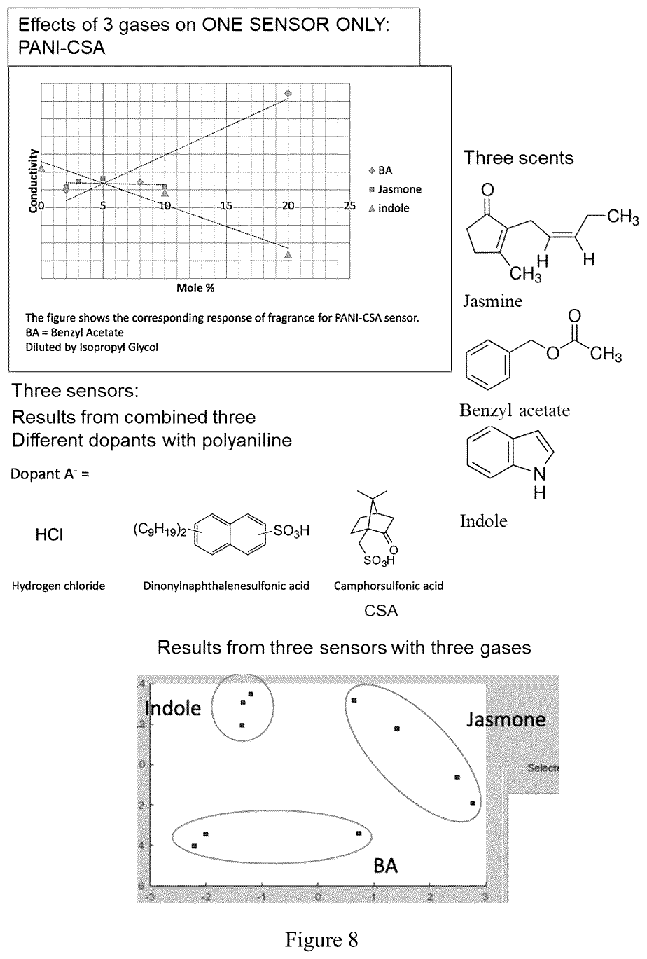

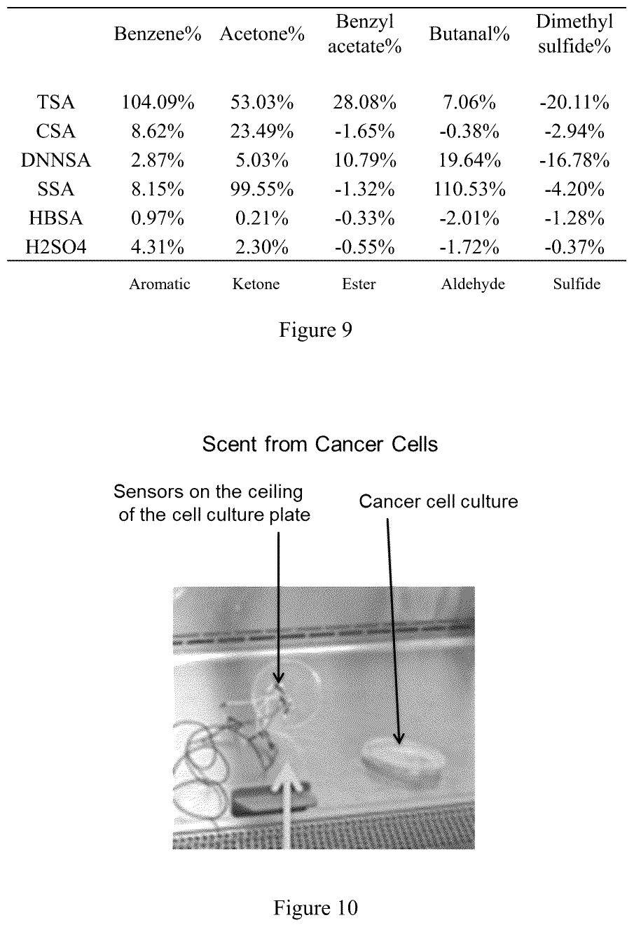

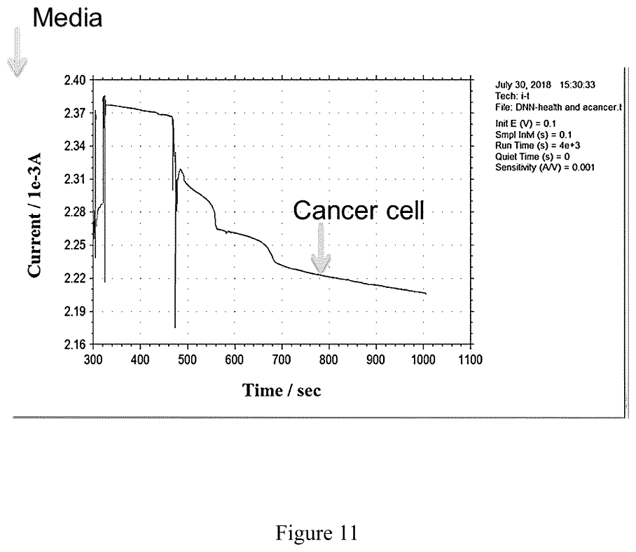

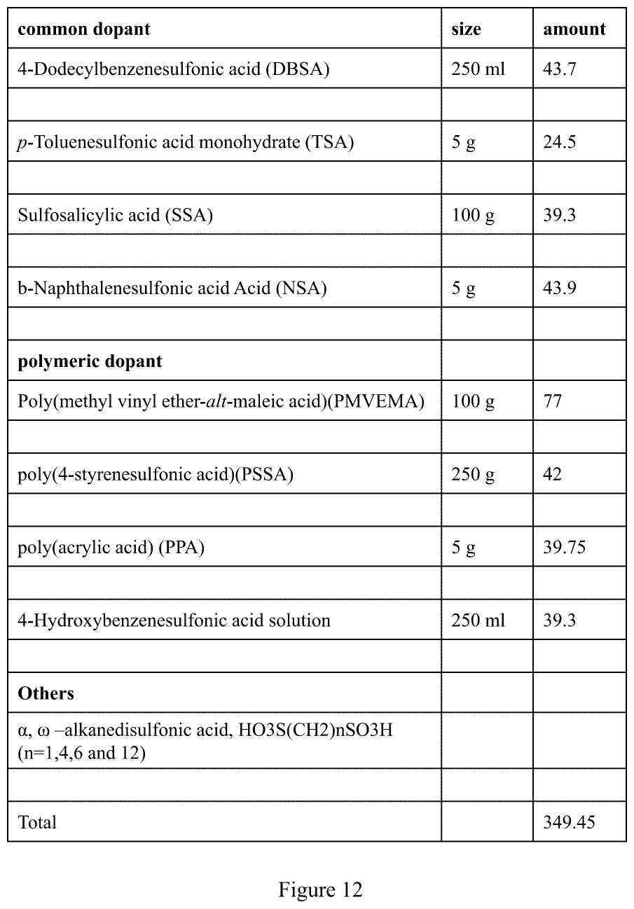

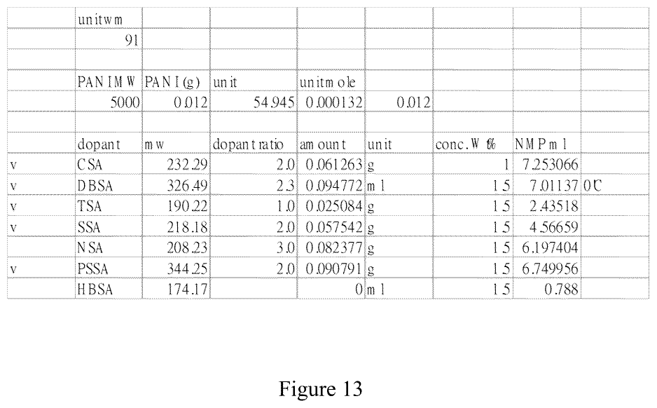

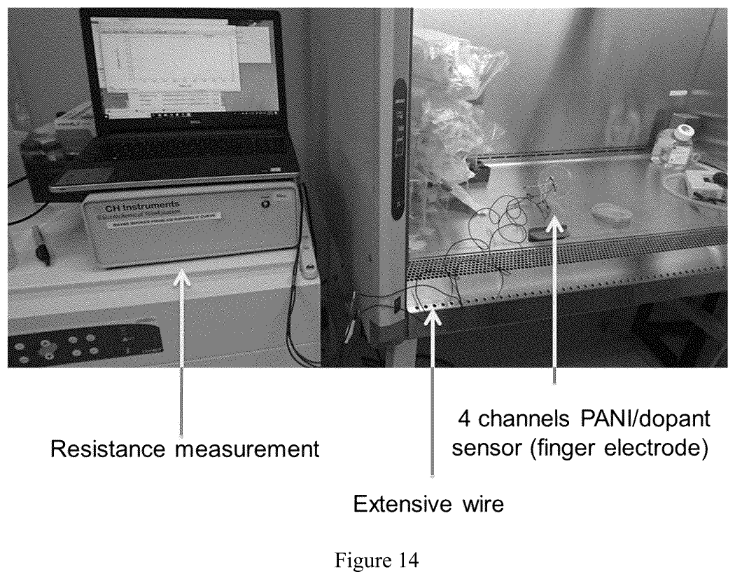

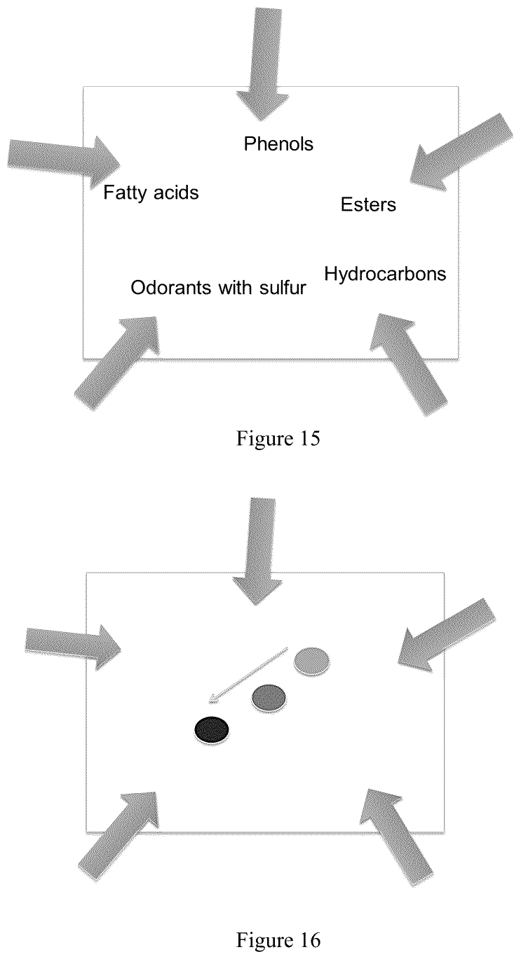

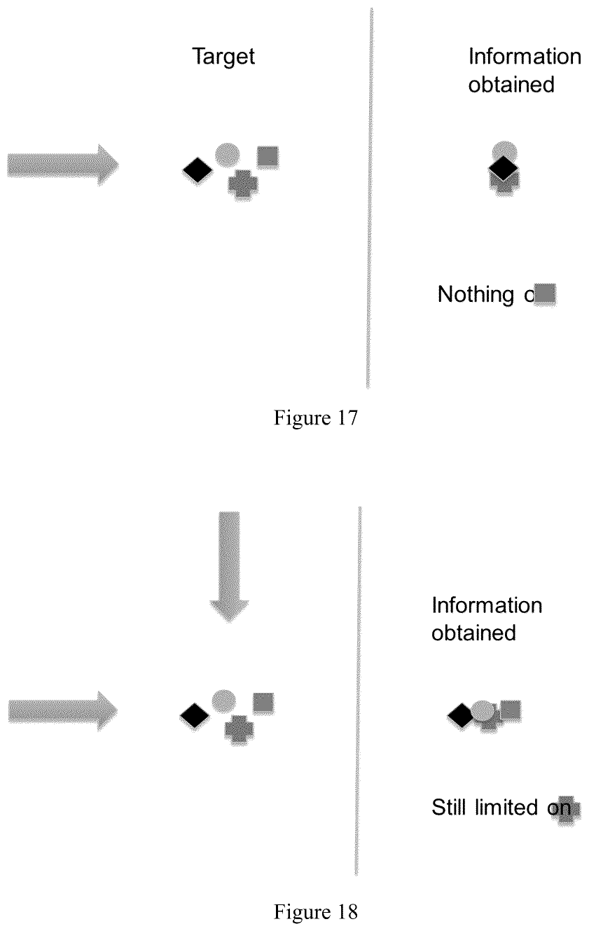

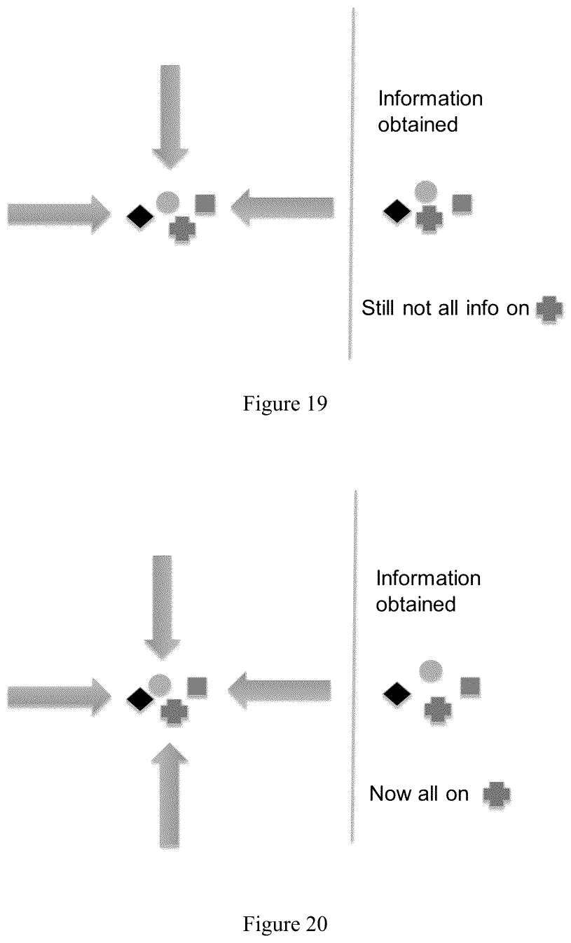

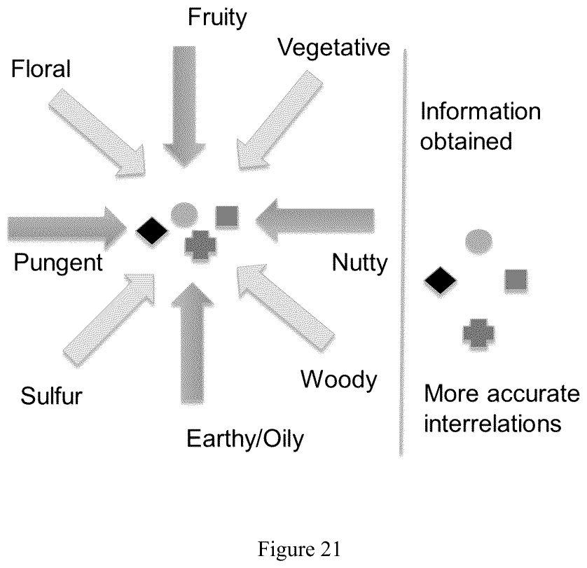

FIGS. 6 through 21 shows data obtained using methods and systems of the present disclosure. The figures described show gas responses on one polyaniline sensor, an example of one sensor with three gases and three sensors on three gases, six sensors on five gases—building the MOLECULAR FRAME, measuring the scent from cancer cells, changing the frame from 5 to four gases, and various other data. See FIG. 8.

Although the present disclosure has been described with respect to one or more particular embodiments and/or examples, it will be understood that other embodiments and/or examples of the present disclosure may be made without departing from the scope of the present disclosure.

1. A method for identifying the presence or absence of a physiological state and/or a disease state by analysis of a test biogas sample from an individual or a biological sample comprising:
providing an array of sensors wherein each sensor of the array comprises an organic semiconducting material, wherein at least two sensors of the array have different organic semiconducting material or have the same semiconducting material at different concentrations, and the oxidation state of the organic semiconducting material of each sensor changes in response to one or more specific volatile compounds, and the at least two sensors can detect distinct volatile compounds or the same volatile compound at different concentrations; and
contacting the array of sensors with the test biogas sample, wherein if the one or more specific volatile compounds are present, the distinct sensors reactive to the specific volatile compounds or a different concentration of the same compound exhibit separately detectable responses and based on the combination of detected responses, the presence or absence of the physiological state and/or disease state is identified.

2. The method of claim 1, wherein the organic semiconducting material is independently for each sensor chosen from organic semiconducting oligomers, organic semiconducting polymers, and combinations thereof, and, optionally, when the organic semiconducting material is an organic semiconducting polymer, the organic semiconducting polymer is modified post polymerization.

3. The method of claim 2, wherein the organic semiconducting material is independently for each sensor chosen from polyaniline (PANI), polythiophene, polypyrrole, substituted analogs thereof, block copolymers comprising one or more block thereof, graft copolymers comprising one or more block thereof, network polymers thereof, and combinations thereof.

4. The method of claim 2, wherein the organic semiconducting oligomers have a molecular weight (M_{w} and/or Mₙ) of 10-500 g/mol, and/or the organic semiconducting polymers have a molecular weight (M_{w} and/or Mₙ) of 200-500,000 g/mol.

5. The method of claim 1, wherein the individual organic semiconducting material(s) is/are amorphous or at least partially crystalline or amorphous.

6. The method of claim 1, wherein the organic semiconducting material(s) further comprise a dopant and the dopant is independently for each sensor chosen from oxidants, acids, polymers, and combinations thereof, or
the organic semiconducting material is a self-doped organic conducting material, copolymers thereof, or network polymers thereof.

7. The method of claim 6, wherein the dopant concentration is independently for each sensor 2 to 50 weight % (based on the total weight of the organic semiconducting material and dopant(s)).

8. The method of claim 1, wherein the array of sensors is a plurality of sensors and the plurality of sensors is provided as a stack.

9. The method of claim 8, wherein the array of sensors comprises one or more stacked sensors.

10. The method of claim 1, wherein the array of sensors are arranged as a planar array and/or vertically stacked array.

11. The method of claim 1, wherein the number of distinct sensors in the array of sensor is chosen from 2 to 1,000.

12. The method of claim 1, wherein one or more of the distinct sensors further comprise one or more polymeric materials other than the organic semiconducting material(s).

13. The method of claim 1, wherein the test biogas sample is breath and/or a gas sample derived from one or more bodily fluid(s), cells, stool, tissue, or a combination thereof.

14. The method of claim 1, further comprising recording the response from the sensor array and generating a pattern based on the response of the plurality of distinct sensors; and comparing the pattern obtained from the test biogas sample to a pattern obtained from a reference biogas sample, which corresponds to a particular physiological or disease state to determine the presence or absence of the physiological state and/or disease state.

15. The method of claim 14, wherein the comparing comprises subjecting the response of the plurality of distinct sensors to a predetermined rule set, wherein the predetermined rule set defines the physiological state(s) and/or disease state(s), thereby identifying the presence or absence of particular physiological state(s) and/or disease state(s), and the comparing optionally comprises utilizing principal component analysis, vector analysis, fuzzy logic, Monte Carlo analysis, or a combination thereof.

16. The method of claim 1, wherein the response is a spectrophotometric response or electronic response.

17. The method of claim 1, wherein the disease state is chosen from cancers, diabetes, autoimmune diseases, mental illnesses, metabolic diseases, and combinations thereof and/or the physiological state is a natural physiological state or an altered physiological state.

18. A system comprising:
a vessel for volatilizing a liquid, the vessel having a vapor outlet;
a channel in communication with the vapor outlet for receiving evaporate from the vessel;
an array of sensors having at least one sensor, arranged as a planar array and/or vertically stacked array, wherein the at least one sensor is configured to contact evaporate in the channel, and wherein each sensor of the at least one sensor comprises:
a first electrical contact;
a second electrical contact; and
the sensors of the array of sensors comprise an organic semiconducting material, wherein at least two sensors have different organic semiconducting material or have the same semiconducting material at different concentrations, and the oxidation state of the organic semiconducting material of each sensor changes in response to one or more specific volatile compounds, and at least two sensors of the array of sensors can detect distinct volatile compounds or the same volatile compound at different concentrations.
